(19) United States
(12) Patent Application Publication
(10) Pub. No.: US 2004/0049170 A1
(43) Pub. Date: Mar. 11, 2004

(54) Balloon-free urinary catheter

(76) Inventor: Robert Adam Snell, Suffolk (GB)

Correspondence Address: David M Carter, Carter DeLuca Farrell & Schmidt, Suite 225, 445 Broad Hollow Road, Melville, NY, 11747, US

(21) Appl. No.: 10/343,651

(22) PCT Filed: Jul. 31, 2001
(86) PCT No.: PCT/GB01/03434

(30) Foreign Application Priority Data
Aug. 3, 2000 (GB) 0019107.2

(51) Int. Cl. A61M 27/00

(52) U.S. Cl. 604/544

(57) ABSTRACT

A balloon-free urinary catheter (15) comprising a tubular retention section (15A) shaped to locate in the neck of a patient's bladder and a tubular drainage section (15B) which, in use, is received in the patient's urethra. The retention section (15A) has a diameter which is normally greater than the neck of the bladder (2) but is stretchable on the application of an axial force thereto to reduce its diameter to less than that of the urethra (3). This allows the catheter to pass through the urethra until the retention section (15A) is positioned inside the bladder where it expands and returns to its original size on release of the axial force and locates in the neck of the bladder to form a seal therewith.

### CROSS-REFERENCE TO RELATED APPLICATION

The present application claims the benefit f priority of International Application No. PCT/GB01/03434 filed on Jul. 31, 2001 which claims the benefit of GB Application No. 0019107.2 filed on Aug. 3, 2000, the entire contents of which are incorporated herein by reference.

### BACKGROUND

1. Technical Field

This invention relates to a balloon-free urinary catheter.

2. Background of Related Art

Many people suffer from a variety of problems with their urinary system ranging from the inability or difficulty to pass urine to urinary incontinence which can be intermittent or chronic. The most common treatment for many of these disorders is to fit a FOLEY catheter to control drainage of urine from the bladder. A FOLEY catheter is a flexible tube, usually made from latex, which has a central lumen and an inflatable balloon incorporated into a wall of its distal end. With the balloon deflated, the catheter tube can be passed through the urethra into the bladder. Once the balloon at the distal end of the tube is located in the bladder, it is inflated and then rests in the neck of the bladder to provide a seal therewith and prevent the tube from being easily removed. A tubular extension extends a few centimetres beyond the top of the balloon and has holes in its wall to allow urine to pass from the bladder into the central lumen tube. It then flows to the proximal end of the device which is fitted with a connector to inflate the balloon and a second connector to close the central lumen. A collection bag is usually connected to the second connector.

The FOLEY catheter was developed over 40 years ago and has many disadvantages. Firstly, about half of all catheterised patients block their catheters. The FOLEY catheter does not allow for easy removal and cleaning or back-flushing and drainage whilst inserted. Furthermore, it does not deal effectively with those patients whose catheters block and most catheterised patients develop infections of some sort when using a FOLEY catheter which in some cases can be problematic. The FOLEY catheter is very obtrusive, particularly for elderly patients requiring long term catheterisation (the largest group) so its use represents a significant degradation in their quality of life. The major advantage of the FOLEY design is that it is relatively cheap to manufacture, although it does require multiple-dip coatings of latex which can be time consuming. It is also widely available and accepted by the medical profession despite its shortcomings.

### SUMMARY

Preferably the retention section is made of a braided material including structural filaments that are arranged at an angle such that when the axial force is applied to the retention section, the tube decreases in diameter but returns to its original shape due to the hoop stress within the braid structure. Preferably, the retention section is made of a polymer such as polyurethane, neoprene, styrene ethylene butadienestyrene (SEBS), styrene butadiene styrene (SBS), plasticised PVC or thermoplastic valcanites. The structural filaments can be made of a ceramic material or metallic wires or polypropylene, nylon, polyurethane, polyethylene. The diameter of the retention section increases on the removal of said axial force until the angle of the braids in relation to the longitudinal axis of the catheter reaches a critical angle. The preferred critical angle is tan⁻¹√2=54.73°.

The tubular drainage section can also be made of a braided material including structural filaments that are arranged at an angle such that when the axial force is applied thereto, the tube decreases in diameter but returns to its original shape due to the hoop stress within the braid structure.

In the preferred embodiment, the braid angle of the filaments in the tubular retention and drainage sections is different so that said tubular sections will expand differentially on release of the axial pressure applied to the retention section.

Preferably the catheter is made of a thin walled elastomeric polymer tubing for example polyurethane, neoprene, styrene ethylene butadiene styrene (SEBS), styrenebutadiene styrene (SBS), plasticised PVC or thermoplastic vulcanites.

Conveniently a valve is located in the proximal end of the catheter which can be of any convenient type.

### BRIEF DESCRIPTION OF THE DRAWINGS

Embodiments of the invention will now be described, by way of example only, with reference to the accompanying drawings, in which

FIG. 1 is a cross section of the area surrounding the urinary tract showing a prior art FOLEY catheter fitted in the bladder;

FIG. 2 is a side view of a urinary catheter of the present invention in its normal relaxed state;

FIG. 3 is a view of the catheter shown in FIG. 2 in its elongated reduced diameter state ready for insertion into the bladder;

FIG. 4 shows the urinary catheter of FIGS. 2 and 3 but in its relaxed normal state when in position in a patients bladder;

FIG. 5 is an end view of the catheter shown in FIGS. 2-4

FIG. 6 is a cross section through the catheter of FIGS. 2-5 but including the placement tool;

FIG. 7 is an end view of the catheter of FIGS. 2-6 but including a return spring in the retention section; and

FIG. 8 shows the catheter of FIG. 4 in position in a patient's bladder.

### DETAILED DESCRIPTION OF PREFERRED EMBODIMENTS

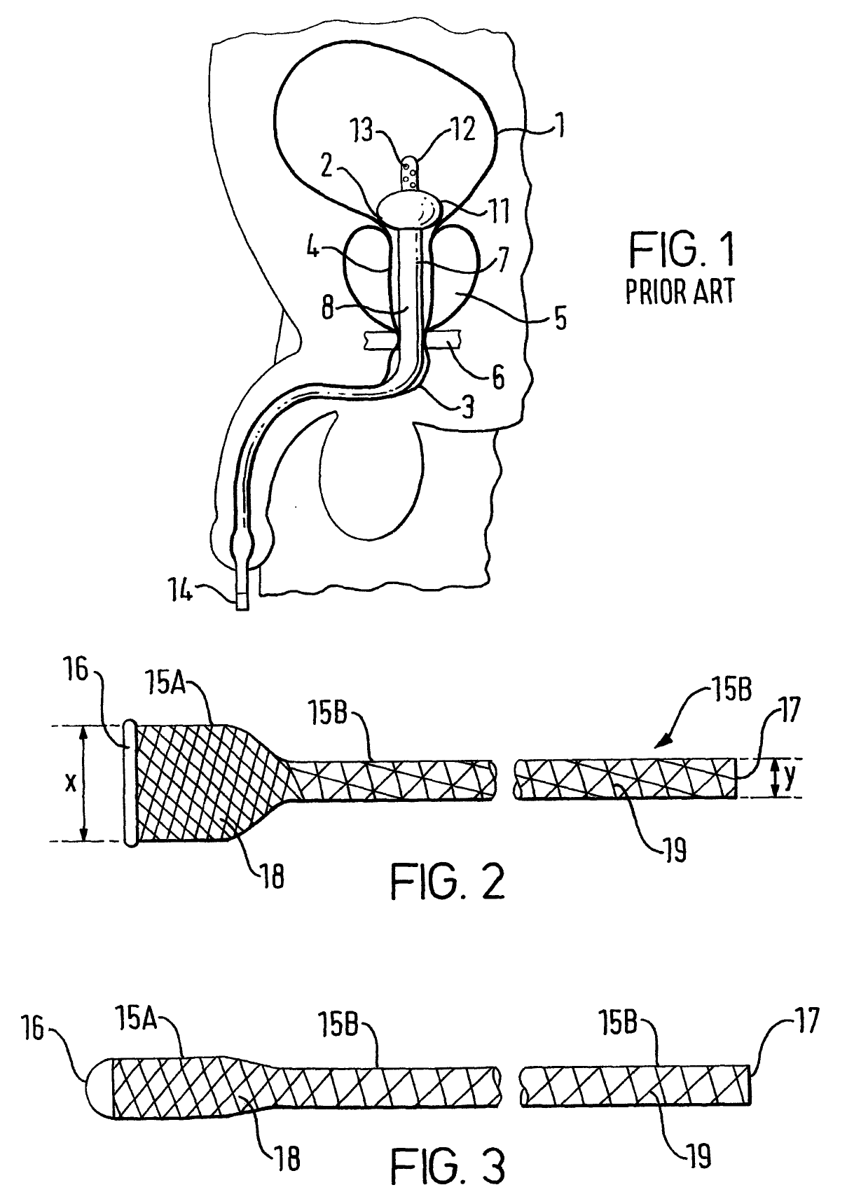

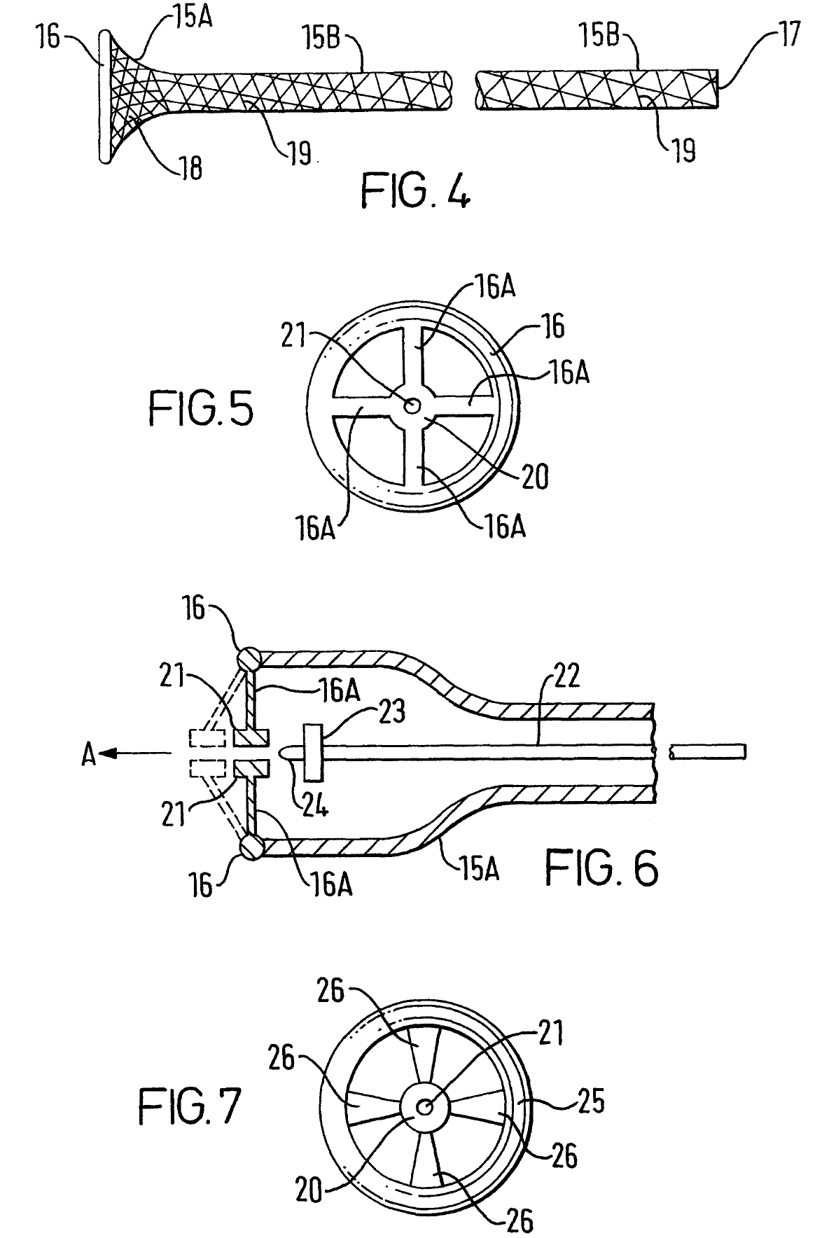

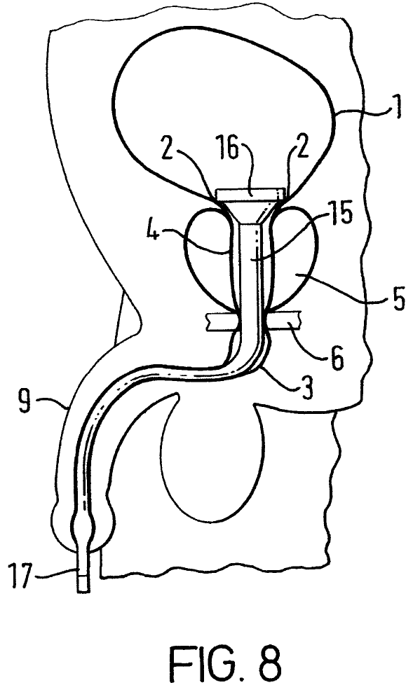

Referring to FIG. 1 of the drawings, there is shown a bladder 1 having a neck 2 which tapers into prostatic part 4 of urethra 3 which is surrounded by prostate gland 5. The external sphincter 6 is also shown whose normal function is to keep the urethra closed until urination when it opens for emptying the bladder 1.

A prior art FOLEY type catheter 7 having an elongate body section 8 and a balloon 11 at one end is seated in the tapered neck 2 of the bladder 1 to make a seal therewith. The balloon 11 has a tubular section 12 extending therefrom with holes 13 therein.

To insert the catheter 7 into the bladder 1, the balloon 11 is collapsed and the catheter 7 is inserted through the urethra 3 until the balloon portion 11 has traveled beyond the tapered neck 2 of the bladder 1. The balloon 11 is then inflated to its condition illustrated in FIG. 1 as a result of which it makes a seal with the tapered neck 2 of the bladder 1. The body portion 8 of the catheter 7 is located within the prostatic part 4 of the urethra 3 and its distal end 14 protrudes just beyond the end of the urethra 3. A valve incorporating a connector (not shown) would normally be attached to the distal end 14. Urine drains from the bladder 1 through the holes 13

19. A balloon-free urinary catheter including a tubular retention section (15A) which, in use, locates in the neck of a patient's bladder, and a tubular drainage section (15B) which, in use, is received in the patient's urethra, the retention section having a diameter which is normally greater than the neck of the bladder but being stretchable on the application of an axial force thereto to reduce its diameter to less than that of the urethra to allow the catheter to travel therethrough until the retention section is positioned inside the bladder, the retention section (15A) being constructed so that it will expand and return to its original size on release of the axial force whereby it locates in the neck of the bladder and engages therewith to form a seal, wherein the improvement comprises:
the retention section (15A) and the drainage section (15B) being both made of a braided material comprising structural filaments arranged at a braid angle so that, when the axial force is applied to the catheter, the retention section (15A) and the drainage section (15B) decrease in diameter and increase once the axial force is removed or reversed.

20. A urinary catheter as claimed in claim 19, wherein the braid angle of each of the retention section (15A) and the drainage section (15B) is different so that the retention section (15A) and the drainage section (15B) will expand differentially on release of the axial force applied to the retention section.

21. A urinary catheter as claimed in claim 20, wherein the retention section (15A) is of a greater diameter than the drainage section (15B).

22. A urinary catheter as claimed in claim 19, further comprising:
locating means (16) incorporated into the distal end of the retention section (15A) against which the axial force can be applied to stretch the retention section and reduce the diameter of the retention section.

23. A urinary catheter as claimed in claim 22, wherein the locating means (16) is made of a shape memory metal.

24. A urinary catheter as claimed in claim 22, wherein the locating means is made of a polymeric material such as nylon, polypropylene, polyethylene, polyearbonate or PTFE.

25. A urinary catheter as claimed in claim 19, further comprising:
spring means incorporated into the retention section (15A) to assist in its recovery to its original diameter on release of the axial force.

26. A urinary catheter as claimed in claim 25, wherein the spring means is radially expandable.

27. A urinary catheter as claimed in claim 26, wherein the spring means is made of a polymer material.

28. A urinary catheter as claimed in claim 27, wherein the spring means is made of one of polypropylene, nylon, polyurethane and polyethylene.

29. A urinary catheter as claimed in claim 19, further comprising:
a flexible bead (16) provided around the distal end of the retention section (15A) to restore the retention section to its original shape on release of the axial force.

30. A urinary catheter as claimed in claim 29, wherein the bead (16) is made of an elastomeric polymer.

31. A urinary catheter as claimed in claim 19, wherein the outer diameter of the tubular retention section (15A), in its relaxed condition, is in the range from about 12-30 mm.

32. A urinary catheter as claimed in claim 19, wherein the outer diameter of the tubular drainage section (15B) is in the range from about 5-12 mm.

33. A urinary catheter as claimed in claim 19, wherein the diameter of the retention section increases on the removal of the axial force until the braid angle in relation to the longitudinal axis of the catheter reaches a critical angle.

34. A urinary catheter as claimed in claim 33, wherein the critical angle is tan⁻¹√2=54.73°.

35. A urinary catheter as claimed in claim 19, wherein the catheter is made of thin walled polyurethane tubing.

36. A urinary catheter as claimed in claim 19, further comprising:
a valve is located in the proximal end of the catheter.

37. A balloon-free urinary catheter comprising:
a tubular retention section (15A) which, in use, locates in the neck of a patient's bladder, the retention section having a diameter which is normally greater than the neck of the bladder but being stretchable on the application of an axial force thereto to reduce the diameter of the retention section to less than that of the urethra to allow the catheter to travel therethrough until the retention section is positioned inside the bladder, the retention section (15A) being constructed so that it will expand and return to its original size on release of the axial force whereby it locates in the neck of the bladder and engages therewith to form a seal; and
a tubular drainage section (15B) which, in use, is received in the patient's urethra, wherein the retention section (15A) and the drainage section (15B) are both made of a braided material including structural filaments arranged at a braid angle so that, when the axial force is applied to the catheter, the retention section (15A) and the drainage section (15B) decrease in diameter and once the axial force is removed or reversed increase in diameter.
